(19) United States
(12) Patent Application Publication
(10) Pub. No.: US 2021/0353733 A1
(43) Pub. Date: Nov. 18, 2021

(54) VACCINE AND ANTIBODY AGAINST CLOSTRIDIOIDES DIFFICILE TOXIN

(71) Applicants: Technische Universität Braunschweig, Braunschweig (DE); Max-Planck-Gesellschaft Zur Förderung Der Wissenschaft E.V., München (DE); Medizinische Hochschule Hannover, Hannover (DE)

(72) Inventors: Viola Fühner, Braunschweig (DE); Michael Hust, Braunschweig (DE); Stefan Dübel, Braunschweig (DE); Felix Löffler, Potsdam (DE); Ralf Gerhard, Hannover (DE)

(21) Appl. No.: 17/277,572

(22) PCT Filed: Sep. 19, 2018
(86) PCT No.: PCT/EP2018/075397

§ 371 Date: Mar. 18, 2021

(51) Int. Cl. A61K 39/08; C07K 16/12

(52) U.S. Cl. CPC A61K 39/08; C07K 16/1282; C07K 2317/76; C07K 2317/565; C07K 2317/567; C07K 2317/34

(57) ABSTRACT

The invention provides an antigenic peptide comprising an epitope for use in the prevention and/or treatment of an infection by C. difficile, e.g. for use as a vaccine in the prevention and/or treatment of an infection by C. difficile, and protective antibodies directed against the epitope.

The present invention relates to a peptide containing a novel peptide epitope for use as antigen, preferably contained in an antigenic peptide, e.g. in the prevention and/or treatment of an infection by Clostridioides difficile (former name: Clostridium difficile). Further, the invention relates to a process for producing and/or selecting at least one antibody directed against the novel peptide epitope and to binding peptides having affinity for the novel peptide epitope, and to the use of such binding peptides in the prevention and/or treatment of an infection by Clostridioides difficile (C. difficile). C. difficile secretes C. difficile Toxin A (TcdA) and C. difficile Toxin B, both of which are peptides, TcdA has 308 kDa, TcdB has 270 kDa. The peptide epitope is a portion of peptide toxin TcdB, and the binding peptide is specific for this portion of TcdB.

### STATE OF THE ART

WO 2016/131157 A1 describes peptides comprising portions of Clostridium difficile toxins A and B, which portions contain epitopes, as well as the generation of antibody directed against the peptides, both for use in the treatment of Clostridium difficile infections.

### OBJECT OF THE INVENTION

It is an object of the invention to provide a peptide containing an alternative epitope suitable for raising an immune response in the prevention or treatment of infections by C. difficile and/or for selecting binding peptides for use of the peptide and for use of the binding peptides in the prevention or treatment of infections by C. difficile.

### DESCRIPTION OF THE INVENTION

The invention achieves the object by the features of the claims, especially by providing an antigenic peptide comprising or consisting of the epitope of SEQ ID NO: 13 for use in the prevention and/or treatment of an infection by C. difficile, e.g. for use as a vaccine in the prevention and/or treatment of an infection by C. difficile. Accordingly, the antigenic epitope can be administered to a mammal, e.g. to a human, for the prevention and/or treatment of an infection by C. difficile, e.g. by injection into the mammal. For use of the antigenic peptide as a vaccine, the antigenic peptide can be formulated in a pharmaceutical composition, e.g. in a mixture with an adjuvant.

The epitope of SEQ ID NO: 13 has been identified as a target of antibody binding, which results in the neutralization of the C. difficile toxin B. The epitope of SEQ ID NO: 13 is based on SEQ ID NO: 1, which is comprised of amino acids No. 402 to 437 of SEQ ID NO: 2, which is the amino acid sequence of TcdB of the reference strain (clade 1) VPI10463, as well as on the epitopes of variants of TcdB, which are SEQ ID NO: 11 for toxin B of hypervirulent strain 820291 (clade 2) containing the epitope at amino acids No. 402 to 437, and SEQ ID NO: 12 1470 (clade 4) containing the epitope at amino acids No. 403 to 438.

Accordingly, SEQ ID NO: 2, SEQ ID NO: 11 and SEQ ID NO: 12 also represent the amino acid sequences of TcdB variants having essentially the same length and functional elements, e.g. peptides having a homology of at least 90%, preferably of at least 95%, more preferably of at least 98% or at least 99%, to SEQ ID NO: 2, SEQ ID NO: 11 and/or to SEQ ID NO: 12.

It has been found that the SEQ ID NO: 13, e.g. SEQ ID NO: 1, amino acids No. 402 to 437 of SEQ ID NO: 11 and/or amino acids No. 403 to 438 of SEQ ID NO: 12, forms a unique epitope, especially a unique antigenic epitope of C. difficile toxin B (TcdB), and that a binding peptide with affinity to this epitope can effectively neutralize TcdB, especially wild-type TcdB containing this epitope. SEQ ID NO: 13 (three letter code) has the following amino acids and one of the amino acids (single letter code) in the place of the amino acid No. for Xaa as indicated below:

| amino | | | | | | | | | | | | | | |
|---|---|---|---|---|---|---|---|---|---|---|---|---|---|---|
| acid No. | 1 | 2 | 3 | 4 | 5 | 6 | 7 | 8 | 9 | 10 | 11 | 12 | 13 | 14 |
| SEQ ID | Gln | Ile | Xaa | Asn | Arg | Tyr | Lys | Ile | Leu | Asn | Xaa | Xaa | Leu | Asn |
| NO: 13 | | | | | | | | | | | | | | |
| possible | | | EQDN | | | | | | | | NDEQ | ST | | |
| amino acids | | | | | | | | | | | | | | |

| amino | | | | | | | | | | | | | |
|---|---|---|---|---|---|---|---|---|---|---|---|---|---|
| acid No. | 15 | 16 | 17 | 18 | 19 | 20 | 21 | 22 | 23 | 24 | 25 | 26 | 27 |
| SEQ ID | Pro | Xaa | Ile | Ser | Xaa | Xaa | Asn | Asp | Phe | Asn | Thr | Thr | Xaa |
| NO: 13 | | | | | | | | | | | | | |
| possible | | AIVL | | | EQDN | DGAVIL | | | | | | | TMS |
| amino acids | | | | | | | | | | | | | |

| amino | | | | | | | | | |
|---|---|---|---|---|---|---|---|---|---|
| acid No. | 28 | 29 | 30 | 31 | 32 | 33 | 34 | 35 | 36 |
| SEQ ID | Asn | Xaa | Phe | Xaa | Xaa | Ser | Xaa | Xaa | Ala |
| NO: 13 | | | | | | | | | |
| possible | | TANSTLVID | | IGLVA | DENQ | | ILVA | MGAVIL | |
| amino acids | | | | | | | | | |

The antigenic peptide comprising the epitope of SEQ ID NO: 13 is especially for use in the prevention or treatment of infections by C. difficile, especially for raising an immune response directed against TcdB, e.g. for neutralizing TcdB. Preferably, the antigenic peptide contains at least two copies of SEQ ID NO: 13.

The complete TcdB peptide contains an N-terminal glucosyltransferase domain (GTD) at amino acids No. 1 to 543 of SEQ ID NO: 2, a cysteine protease domain (CPD) at amino acids No. 544 to 767 of SEQ ID NO: 2. The CPD catalyses proteolytic auto-processing of TcdB to release the GTD into the cytosol of an infected cell upon translocation. Amino acids No. 768 to 1852 of SEQ ID NO: 2 are currently designated a translocation domain (TLD) and contain three cell surface binding regions. At the C-terminus, TcdB contains so-called combined repetitive oligo peptides (CROPs) which are currently assumed to have a cell-binding and stabilizing role.

The antigenic peptide comprising the epitope of SEQ ID NO: 13 is not the complete TcdB peptide, and it preferably does not comprise an E3 domain and/or no E4 domain, e.g. no CROPs. Optionally, in addition or in the alternative, the antigenic peptide does not contain a functional GTD and/or does not comprise a functional TLD and/or does not comprise a functional CPD and/or does not comprise CROPs, preferably the antigenic peptide does not comprise a functional GTD, no functional CPD and no functional CROPs, e.g. in order to prevent a toxic effect of the antigenic peptide.

The E3 domain is comprised of amino acids No. 2138 to 2271 of SEQ ID NO: 2, the E4 domain is comprised of amino acids No. 2272 to 2366 of SEQ ID NO: 2. The CROPs comprises domains E1, E2, E3 and E4, wherein the E1 domain is comprised of amino acids No. 1875 to 2005 of SEQ ID NO: 2, the E2 domain is comprised of amino acids No. 2006 to 2137 of SEQ ID NO: 2. The domains E1 to E4 of SEQ ID NO: 11 and SEQ ID NO: 12, respectively, are comprised of the same amino acid Nos. The CROPs is comprised of amino acid Nos. 1853 to 2366 of SEQ ID NO: 2, of SEQ ID NO: 11 and of SEQ ID NO: 12

For the purpose of the present invention, the term peptide is not limited to a certain length of the amino acid chain.

The antigenic peptide comprising the epitope of SEQ ID NO: 13 can e.g. contain only the portion of the GTD consisting of amino acids No. 361 to 543 of SEQ ID NO: 2, which portion is given as SEQ ID NO: 3, or a fraction thereof, or can contain only the portion of the GTD including the CPD, the fraction consisting of amino acids No. 361 to 767 of SEQ ID NO: 2, which portion is given as SEQ ID NO: 4, or a fraction thereof, and optionally can contain additional amino acids, or consist of one of these portions. These portions exclude the catalytically active centre of the GTD and therefore avoid a toxic effect of the GTD. The antigenic peptide comprising the epitope of SEQ ID NO: 13 preferably does not comprise amino acids No. 322 to 325 and/or does not comprise amino acids No. 340 to 351, e.g. does not comprise amino acids No. 290 to 360 or amino acids No. 322 to 351 of SEQ ID NO: 2, resp. of SEQ ID NO: 11 or of SEQ ID NO: 12.

Further, the antigenic peptide comprising the epitope of SEQ ID NO: 13 preferably does not comprise the amino acid sequence of the CPD, and/or not the amino acid sequence for the TLD and/or not the amino acid sequence of the CROPs.

On the basis of the epitope of SEQ ID NO: 13, the invention also relates to a process for eliciting binding peptides, e.g. antibody, and/or for generating and/or isolating binding peptides having affinity for the epitope of SEQ ID NO: 13. A process for eliciting and/or generating binding peptides, e.g. antibodies, in an animal, preferably in a mammal, e.g. a human, comprises the steps of administering an antigenic peptide containing the epitope of SEQ ID NO: 13, e.g. in a peptide which in addition to SEQ ID NO: 13 contains amino acid sections which are not antigenic to the animal or which peptide consists of SEQ ID NO: 13, preferably in a pharmaceutical composition containing an adjuvant, preferably followed by at least one booster administration of the antigenic peptide containing the epitope of SEQ ID NO: 13 to the animal.

Subsequently, antibody-producing cells can be isolated from the animal, e.g. isolating B-cells from blood, or isolating spleen cells, for generating antibody-producing cells, e.g. using the hybridoma technique. For example, B-cell sorting from a biopsy, e.g. whole blood, obtained from a human, e.g. who is diagnosed to have an infection by C. difficile, and identification of antibody or cells producing antibody binding to a peptide containing the epitope of SEQ ID NO: 13 can be used.

As a further alternative, phage display, e.g. as described herein, using a library e.g. of antibodies or of arbitrary peptides on an antigenic peptide comprising the epitope of SEQ ID NO: 13 can be used for selection of binding peptides. Optionally, antibody can be isolated from the serum of the animal to which the peptide containing the epitope of SEQ ID NO: 13 was administered, e.g. by immobilizing a peptide containing the epitope of SEQ ID NO: 13 and contacting this with the serum, washing the immobilized peptide and eluting antibody from the immobilized peptide to generate an isolated fraction of antibody having affinity for the epitope of SEQ ID NO: 13. As a representative epitope, SEQ ID NO: 1 can be used.

Administration of an antigenic peptide comprising the epitope SEQ ID NO: 13, e.g. as the only antigenic epitope with N-terminal and/or C-terminal additional amino acid sections that can be devoid of antigenic epitopes, to an experimental animal leads to the generation of antibody which recognize the epitope of SEQ ID NO: 13.

A binding peptide that has affinity for the epitope SEQ ID NO: 13 neutralizes the effect of the TcdB peptide, showing that a binding peptide recognizing the epitope SEQ ID NO: 13 confers at least partial prevention and/or at least partial treatment or cure of an infection by C. difficile. This neutralizing effect of binding peptides directed against the epitope of SEQ ID NO: 13 is shown e.g. by cell assays detecting reduction of cell rounding in the presence of TcdB.

The epitope of SEQ ID NO: 13 was identified on fragments of the TcdB peptide by two scFv-Fc molecules selected from a naïve human phage display library as scFv. From the phage library, two binding peptides were identified that have a high binding affinity for the epitope SEQ ID NO: 1, which was used as a representative of SEQ ID NO: 13.

Generally, the binding peptides of the invention have affinity for the epitope of SEQ ID NO: 1 and the binding peptides can have a format of natural or synthetic binding peptides, e.g. of antibodies, especially IgG, IgM, IgA or IgE, scFv, scFv-Fc, minibodies, diabodies or other antibody derived formats.

In detail, the binding peptide of the invention has a paratope of framework regions (FR) and of complementarity determining regions (CDR) in an arrangement of FR1-CDR1-FR2-CDR2-FR3-CDR3, preferably additionally FR4,

in a first embodiment preferably with the following heavy chain complementarity determining regions (CDRH):
CDRH1 consisting of amino acids 26 to 33 of SEQ ID NO: 5,
CDRH2 consisting of amino acids 51 to 58 of SEQ ID NO: 5, and
CDRH3 consisting of amino acids 97 to 112 of SEQ ID NO: 5,
preferably with FR1 consisting of amino acids 1 to 25 of SEQ ID NO: 5,
FR2 consisting of amino acids 34 to 50 of SEQ ID NO: 5,
FR3 consisting of amino acids 59 to 96 of SEQ ID NO: 5,
and optional FR4 consisting of amino acids 113 to 123 of SEQ ID NO: 5,
preferably in combination with the following light chain complementarity determining regions (CDRL):
CDRL1 consisting of amino acids 26 to 34 of SEQ ID NO: 6,
CDRL2 consisting of amino acids 52 to 54 of SEQ ID NO: 6,
CDRL3 consisting of amino acids 91 to 101 of SEQ ID NO: 6,
preferably with FR1 consisting of amino acids 1 to 25 of SEQ ID NO: 6,
FR2 consisting of amino acids 35 to 51 of SEQ ID NO: 6,
FR3 consisting of amino acids 55 to 90 of SEQ ID NO: 6,
and optional FR4 consisting of amino acids 102 to 111 of SEQ ID NO: 6.

It has been found that the heavy chain can form a paratope also with another light chain, e.g. the heavy chain of the first embodiment can associate with the light chain of the second embodiment, and the heavy chain of the second embodiment can associate with the light chain of the first embodiment. Optionally, the heavy chain can be present in combination with another light chain, as the specificity for the antigen is mainly determined by the heavy chain.

In the first embodiment, the binding peptide can comprise the heavy chain of SEQ ID NO: 5 and the light chain of SEQ ID NO: 6, e.g. with a linker between the heavy chain and the light chain. SEQ ID NO: 9 is a binding peptide containing the heavy chain-linker-light chain of the first embodiment, wherein the linker is comprised of amino acids 124 to 141 as an exemplary scFv.

In a second embodiment, the binding peptide, which preferably is an antibody, preferably has the following heavy chain complementarity determining regions (CDRH):

CDRH1 consisting of amino acids 26 to 33 of SEQ ID NO: 7,
CDRH2 consisting of amino acids 51 to 58 of SEQ ID NO: 7, and
CDRH3 consisting of amino acids 97 to 117 of SEQ ID NO: 7,
preferably with FR1 consisting of amino acids 1 to 25 of SEQ ID NO: 7,
FR2 consisting of amino acids 34 to 50 of SEQ ID NO: 7,
FR3 consisting of amino acids 59 to 96 of SEQ ID NO: 7,
and optional FR4 consisting of amino acids 118 to 128 of SEQ ID NO: 7,
preferably in combination with the following light chain complementarity determining regions (CDRL):
CDRL1 consisting of amino acids 25 to 33 of SEQ ID NO: 8,
CDRL2 consisting of amino acids 51 to 53 of SEQ ID NO: 8,
CDRL3 consisting of amino acids 90 to 100 of SEQ ID NO: 8,
preferably with FR1 consisting of amino acids 1 to 24 of SEQ ID NO: 8,
FR2 consisting of amino acids 34 to 50 of SEQ ID NO: 8,
FR3 consisting of amino acids 54 to 89 of SEQ ID NO: 8,
and optional FR4 consisting of amino acids 101 to 110 of SEQ ID NO: 8.

In the second embodiment, the binding peptide can comprise the heavy chain of SEQ ID NO: 7 and the light chain of SEQ ID NO: 8, e.g. with a linker between the heavy chain and the light chain. SEQ ID NO: 10 is a binding peptide containing the heavy chain-linker-light chain of the first embodiment, wherein the heavy chain is comprised of amino acids 1-128, the light chain is comprised of amino acids 147-256, and the linker is comprised of amino acids 129 to 146 as an exemplary scFv.

In detail, the epitope of SEQ ID NO: 13, e.g. represented by SEQ ID NO: 1, and the binding peptide of the first and second embodiments were selected by a phage display library using the naïve human antibody gene libraries HAL9 and HAL10, against immobilized peptides which was full-length TcdB peptide (SEQ ID NO: 2) or fragments thereof (amino acids 1-1852, amino acids 1-1128, respectively of SEQ ID NO: 2), each with an additional C-terminal His₆ tag, expressed in Bacillus megaterium. Fusion peptides that were His-tagged TcdB or fragments thereof were isolated by Ni²⁺ affinity chromatography (Ni-IDA columns, Macherey-Nagel, Germany). The phage display library containing the antibody gene libraries, which can also be described as binding peptide libraries, herein is also termed antibody phage library.

The antibody phage display library contained the binding peptides in scFv format. The library was used for selection by incubation on full-length TcdB and/or on the fragments, immobilized on Costar High binding microtiter plates at room temperature. Negative selection on immobilized peptide of amino acids No. 1-1128 of SEQ ID NO: 2 was used to isolate peptides binding to the TLD. After three rounds of panning, monoclonal scFv were produced and screened for binding to TcdB by an antigen-ELISA. DNA of binding phage was isolated and sequenced, unique scFv sequences of binding peptides were recloned (pCSE2.6-hIgG1-Fc-XP, accessible at PMID:23802841) for production in mammalian cells (HEK293-6E cells) as scFv-Fc, which is an IgG-like bivalent format. Binding peptides were purified using protein A.

For epitope mapping, a phage display library of fragments of the TcdB gene was constructed using Phusion DNA polymerase with tcdb gene specific primers on genomic DNA of C. difficile in PCR and fragmenting the amplificate by ultrasound, filling cohesive ends to blunt ends and phosphorylation by the Fast DNA Repair Kit, obtainable from Thermo Scientific, and cloning the fragments into a dephosphorylated pHORF3 library vector (Kügler et al., Applied Microbiology and Biotechnology, 447-458 (2008)). The vector was used to transform TOP10F′ E. coli, from which the phage library was produced using Hyperphage. Phage displaying toxin fragments were selected on antibody using SEQ ID NO: 9, respectively SEQ ID No: 10. Sequencing of the selected phagemids resulted in identification of TcdB-fragments that were bound by the antibodies.

Further, the epitope was mapped using a 15-mer peptide array with an offset of 2 amino acids for neighbouring peptides.

These assays indicate affinity of the binding peptides, especially of the binding peptides of the first and second embodiments, for the epitope of SEQ ID NO: 13, as exemplified by SEQ ID NO: 1.

The protective effect of antibody having affinity for the epitope of SEQ ID NO: 1 was analysed in an in vitro TcdB neutralization assay using Vero cells (African green monkey kidney cells). Vero cells were cultivated in RPMI medium supplemented with 2 g/L NaHCO₃, 2 mM stable glutamine (FG1215, obtainable from Biochrom) and 10% fetal calf serum at 37° C. in a 5% CO₂ atmosphere, with passaging two to three times per week when confluency exceeded 90%. Presence of the toxin TcdB leads to a breakdown of the actin cytoskeleton, which is easily visible as rounding of the cells using bright field microscopy. Accordingly, activity of the toxin, and the neutralization of the toxin by binding peptide was observed using the microscopic determination of cell rounding. Cells were seeded to 10 000 cells per well of a 96-well cell culture plate (Cellstar, Greiner bio-one, Germany) and cultivated in the medium for 16 h prior to adding TcdB. TcdB was added in an amount to obtain a concentration of 0.1 pM in the cell culture. Before adding the toxin to the cultivated cells, TcdB was diluted in cell culture medium. For positive intoxication assays, the TcdB in medium was added to the cultivated cells. As a negative comparison without intoxication, only medium was added to cultivated cells. For neutralization assays, the binding peptide was added to the TcdB in the medium to a final concentration of 100 nM, incubated for 30 min at room temperature, and then added to the cultivated cells. Cell rounding was analyzed when in the positive intoxication assay 70-80% of cells were rounded, indicating intoxication. For evaluation, the percentage of rounded cells in the neutralization assays was determined and normalized to the percentage of rounded cells determined in the negative and positive comparative intoxication assays.

On the example of the binding peptides of the first and second embodiments, the TcdB neutralization assay showed that the binding peptides of the invention result in an effective neutralization of the full-length toxin TcdB. In this in vitro assay, these exemplary binding peptides were found to neutralize TcdB to more than 75% as indicated by a reduction of rounded cells by more than 75%.

Generally optionally, the peptide comprising the epitope of SEQ ID NO: 13, e.g. of SEQ ID NO: 1, especially when for use as an antigenic peptide, it can comprise additional antigenic epitopes of TcdB and/or of TcdA, e.g. the epitopes of amino acid Nos. 322 to 325 and/or of amino acid Nos. 340 to 351, and/or the domain of amino acid Nos. 290 to 360, resp., of SEQ ID NO: 2 or of SEQ ID NO: 11. Optionally, the antigenic peptide can consist of at least one copy or at least two copies of the epitope of SEQ ID NO: 13 in combination with one or at least two of the epitopes of amino acid Nos. 322 to 325 and/or of amino acid Nos. 340 to 351, and/or the domain of amino acid Nos. 290 to 360 of SEQ ID NO: 2 or of SEQ ID NO: 11.

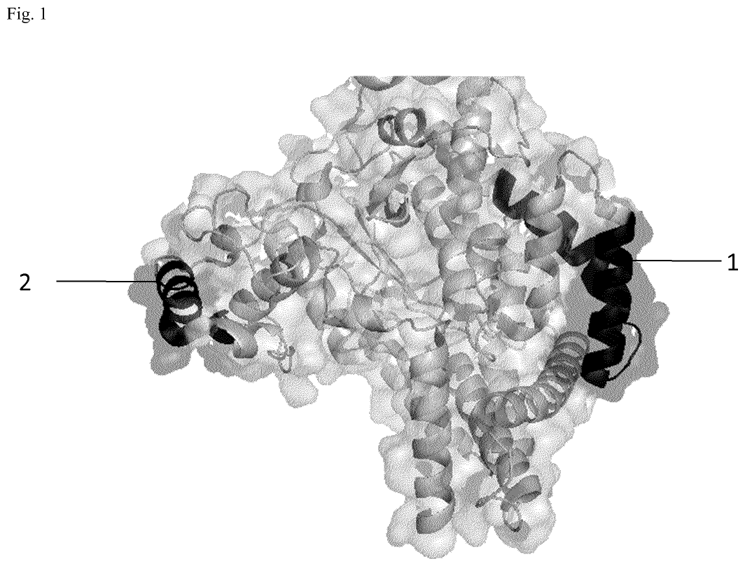

For illustration, FIG. 1 shows a section of a crystal structure of the GT domain of TcdB, modified from a PDB file (accession No 2bvm), indicating at 1 the epitope of SEQ ID NO: 1 emphasized in black, and at 2 indicating amino acids 322-325 and 340-351 also emphasized in black. This shows that the epitope of the invention, and hence the binding site for binding peptides of the invention, are located at a great distance from an epitope indicated at 2 of amino acids 322-325 and 340-351.

1. Antigenic peptide for use in the treatment or prevention of an infection by Clostridioides difficile, the peptide comprising the epitope of SEQ ID NO: 13, which peptide is devoid of an amino acid sequence of amino acids No. 2138 to 2271 of SEQ ID NO: 2 and/or is devoid of amino acids No. 2272 to 2366 of SEQ ID NO: 2.

2. Antigenic peptide for use in the treatment or prevention of an infection by Clostridioides difficile according to claim 1, wherein the peptide is devoid of the amino acid sequence of the CPD and of the amino acid sequence of the CROPs of a sequence having at least 90% homology to SEQ ID NO: 2.

3. Antigenic peptide for use in the treatment or prevention of an infection by Clostridioides difficile according to claim 1, wherein the peptide does not contain one or more of the group consisting of a catalytically active GTD, a functional TLD, a catalytically active CPD, and the CROPs.

4. Antigenic peptide for use in the treatment or prevention of an infection by Clostridioides difficile according to claim 1, wherein the antigenic peptide as a continuous segment of Clostridioides difficile toxin B comprises only the fraction of amino acids of SEQ ID NO: 3 or SEQ ID NO: 4, wherein the peptide optionally contains further epitopes of Clostridioides difficile toxin A and/or toxin B, which further epitopes are contained in segments which do not contain a catalytically active GTD, nor a catalytically active CPD, nor the CROPs.

5. Antigenic peptide for use in the treatment or prevention of an infection by Clostridioides difficile according to claim 1, wherein the antigenic peptide does not contain additional epitopes of Clostridioides difficile toxin B.

6. Antigenic peptide for use in the treatment or prevention of an infection by Clostridioides difficile according to claim 1, comprising at least two copies of the epitope of SEQ ID NO: 13.

7. Antigenic peptide for use in the treatment or prevention of an infection by Clostridioides difficile according to claim 1, comprising at least one or at least two of the epitopes of amino acid Nos. 322 to 325 and/or of amino acid Nos. 340 to 351, and/or the domain of amino acid Nos. 290 to 360 of SEQ ID NO: 2 or of SEQ ID NO: 11.

8. Binding peptide for use in the treatment or prevention of an infection by Clostridioides difficile, the peptide comprising a paratope of framework regions (FR) and of complementarity determining regions (CDR) in an arrangement of FR1-CDR1-FR2-CDR2-FR3-CDR3, preferably additionally FR4, wherein the heavy chain complementarity determining regions (CDRH) are
CDRH1 consisting of amino acids 26 to 33 of SEQ ID NO: 5,
CDRH2 consisting of amino acids 51 to 58 of SEQ ID NO: 5,
CDRH3 consisting of amino acids 97 to 112 of SEQ ID NO: 5,
or
CDRH1 consisting of amino acids 26 to 33 of SEQ ID NO: 7,
CDRH2 consisting of amino acids 51 to 58 of SEQ ID NO: 7, and
CDRH3 consisting of amino acids 97 to 117 of SEQ ID NO: 7.

9. Binding peptide for use according to claim 8, wherein the
FR1 consists of amino acids 1 to 25 of SEQ ID NO: 5,
FR2 consists of amino acids 34 to 50 of SEQ ID NO: 5,
FR3 consists of amino acids 59 to 96 of SEQ ID NO: 5,
and optional FR4 consists of amino acids 113 to 123 of SEQ ID NO: 5,
or
FR1 consists of amino acids 1 to 25 of SEQ ID NO: 7,
FR2 consists of amino acids 34 to 50 of SEQ ID NO: 7,
FR3 consists of amino acids 59 to 96 of SEQ ID NO: 7,
and optional FR4 consists of amino acids 118 to 128 of SEQ ID NO: 7

10. Binding peptide for use according to claim 8, comprising a light chain of FR1-CDR1-FR2-CDR2-FR3-CDR3, preferably additionally FR4, with the light chain complementarity determining regions (CDRL)
CDRL1 consisting of amino acids 26 to 34 of SEQ ID NO: 6,
CDRL2 consisting of amino acids 52 to 54 of SEQ ID NO: 6,
CDRL3 consisting of amino acids 91 to 101 of SEQ ID NO: 6,
or
CDRL1 consisting of amino acids 25 to 33 of SEQ ID NO: 8,
CDRL2 consisting of amino acids 51 to 53 of SEQ ID NO: 8,
CDRL3 consisting of amino acids 90 to 100 of SEQ ID NO: 8.

11. Binding peptide for use according to claim 10, wherein the framework regions (FR) of the light chain are
FR1 consisting of amino acids 1 to 25 of SEQ ID NO: 6,
FR2 consisting of amino acids 35 to 51 of SEQ ID NO: 6,
FR3 consisting of amino acids 55 to 90 of SEQ ID NO: 6,
and optional FR4 consisting of amino acids 102 to 111 of SEQ ID NO: 6
or
FR1 consisting of amino acids 1 to 24 of SEQ ID NO: 8,
FR2 consisting of amino acids 34 to 50 of SEQ ID NO: 8,
FR3 consisting of amino acids 54 to 89 of SEQ ID NO: 8,
and optional FR4 consisting of amino acids 101 to 110 of SEQ ID NO: 8.

12. Binding peptide for use according to claim 8, wherein the paratope is contained in an IgG, IgM, IgA or IgE, scFv, scFv-Fc, minibody or diabody.

13. Process for producing and/or selecting binding peptides directed against Clostridioides difficile toxin B, characterized by the use of an antigenic peptide according to claim 1.


SEQUENCE LISTING (13 sequences)

SEQ ID NO: 1 - protein, 36 aa, Artificial Sequence
  immunogenic epitope of TcdB
QIENRYKILNNSLNPAISEDNDFNTTTNTFIDSIMA

SEQ ID NO: 2 - protein, 2366 aa, Clostridioides difficile
  CHAIN 1..543: GTD domain
  Toxin B of VPI10463 (clade 1)
  CHAIN 402..437: epitope
  CHAIN 544..767: CPD domain
  CHAIN 768..1852: TLD domain
  CHAIN 1853..2366: CROPs
  CHAIN 1875..2005: E1 domain
  CHAIN 2006..2137: E2 domain
  CHAIN 2138..2271: E3 domain
  CHAIN 2272..2366: E4 domain
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKDEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGNTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 3 - protein, 183 aa, Artificial Sequence
  fragment of toxin B
SSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSL

SEQ ID NO: 4 - protein, 407 aa, Artificial Sequence
  fragment of toxin B
SSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEES

SEQ ID NO: 5 - protein, 123 aa, Artificial Sequence
  CHAIN 1..25: FR1
  heavy chain
  CHAIN 26..33: CDRH1
  CHAIN 34..50: FR2
  CHAIN 51..58: CDRH2
  CHAIN 59..96: FR3
  CHAIN 97..112: CDRH3
  CHAIN 113..123: FR4
QVQLQQSGAEVKKPGASVTVSCKASGYTFTNYAIHWVRQAPGQRLEWMGWINPGNGNTKYSQTFQGRVTISRDTSATTASMELSSLRSEDTAVYYCARVIRPSVIVTTPFDFWGQGTLVTVSS

SEQ ID NO: 6 - protein, 111 aa, Artificial Sequence
  CHAIN 1..25: FR1
  light chain
  CHAIN 26..34: CDRL1
  CHAIN 35..51: FR2
  CHAIN 52..54: CDRL2
  CHAIN 55..90: FR3
  CHAIN 91..101: CDRL3
  CHAIN 102..111: FR4
QSALTQPASVSGSPGQSITISCTGTNSDIGGHNYVSWYQQHPGKAPKLIIYDVSNRPSGVSNRFSGSKSGNTASLTISGLQAEDEADYFCGSYTSRGARYVFGGGTKVTVL

SEQ ID NO: 7 - protein, 128 aa, Artificial Sequence
  CHAIN 1..25: FR1
  heavy chain
  CHAIN 26..33: CDRH1
  CHAIN 34..50: FR2
  CHAIN 51..58: CDRH2
  CHAIN 59..96: FR3
  CHAIN 97..117: CDRH3
  CHAIN 118..128: FR4
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDLVFQGRFLEWLSPYYFDYWGQGTLVTVSS

SEQ ID NO: 8 - protein, 110 aa, Artificial Sequence
  CHAIN 1..24: FR1
  light chain
  CHAIN 25..33: CDRL1
  CHAIN 34..50: FR2
  CHAIN 51..53: CDRL2
  CHAIN 54..89: FR3
  CHAIN 90..100: CDRL3
  CHAIN 101..110: FR4
QAVLTQPPSASGTPGQRVAISCSGSSSNIGSNTVNWYQQLPGEAPRLLIYAKNQRPSGVPDRFSASKSGSSASLAITGLQSEDEADYYCATWDDSLSAWVFGGGTKVTVL

SEQ ID NO: 9 - protein, 272 aa, Artificial Sequence
  CHAIN 1..123: heavy chain
  scFv
  CHAIN 124..141: linker
  CHAIN 142..252: light chain
QVQLQQSGAEVKKPGASVTVSCKASGYTFTNYAIHWVRQAPGQRLEWMGWINPGNGNTKYSQTFQGRVTISRDTSATTASMELSSLRSEDTAVYYCARVIRPSVIVTTPFDFWGQGTLVTVSSGSASAPKLEEGEFSEARVQSALTQPASVSGSPGQSITISCTGTNSDIGGHNYVSWYQQHPGKAPKLIIYDVSNRPSGVSNRFSGSKSGNTASLTISGLQAEDEADYFCGSYTSRGARYVFGGGTKVTVLSQPKANPTVTLFPPSSAAAS

SEQ ID NO: 10 - protein, 276 aa, Artificial Sequence
  CHAIN 1..128: heavy chain
  scFv
  CHAIN 129..146: linker
  CHAIN 147..256: heavy chain
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDLVFQGRFLEWLSPYYFDYWGQGTLVTVSSGSASAPKLEEGEFSEARVQAVLTQPPSASGTPGQRVAISCSGSSSNIGSNTVNWYQQLPGEAPRLLIYAKNQRPSGVPDRFSASKSGSSASLAITGLQSEDEADYYCATWDDSLSAWVFGGGTKVTVLGQPKAAPSVTLFPPSSAAAS

SEQ ID NO: 11 - protein, 2366 aa, Clostridioides difficile
  CHAIN 1..543: GTD domain
  Toxin B R20291 (clade 2)
  CHAIN 402..437: epitope
  CHAIN 544..767: CPD domain
  CHAIN 768..1852: TLD domain
  CHAIN 1853..2366: CROPs
  CHAIN 1875..2005: E1 domain
  CHAIN 2006..2137: E2 domain
  CHAIN 2138..2271: E3 domain
  CHAIN 2272..2366: E4 domain
MSLVNRKQLEKMANVRFRVQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTIVESATNDTLESFRENLNDPRFDYNKFYRKRMEIIYDKQKNFINYYKTQREENPDLIIDDIVKIYLSNEYSKDIDELNSYIEESLNKVTENSGNDVRNFEEFKGGESFKLYEQELVERWNLAAASDILRISALKEVGGVYLDVDMLPGIQPDLFESIEKPSSVTVDFWEMVKLEAIMKYKEYIPGYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNAFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKKNYFEGSLGEDDNLDFSQNTVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGLDVDSLSTEIETAIDLAKEDISPKSIEINLLGCNMFSYSVNVEETYPGKLLLRVKDKVSELMPSISQDSIIVSANQYEVRINSEGRRELLDHSGEWINKEESIIKDISSKEYISFNPKENKIIVKSKNLPELSTLLQEIRNNSNSSDIELEEKVMLAECEINVISNIDTQVVEGRIEEAKSLTSDSINYIKNEFKLIESISDALYDLKQQNELEESHFISFEDILETDEGFSIRFIDKETGESIFVETEKAIFSEYANHITEEISKIKGTIFDTVNGKLVKKVNLDATHEVNTLNAAFFIQSLIEYNSSKESLSNLSVAMKVQVYAQLFSTGLNTITDAAKVVELVSTALDETIDLLPTLSEGLPVIATIIDGVSLGAAIKELSETSDPLLRQEIEAKIGIMAVNLTAATTAIITSSLGIASGFSILLVPLAGISAGIPSLVNNELILRDKATKVVDYFSHISLAESEGAFTSLDDKIMMPQDDLVISEIDFNNNSITLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPVITTEYIREKLSYSFYGSGGTYALSLSQYNMNINIELNENDTWVIDVDNVVRDVTIESDKIKKGDLIENILSKLSIEDNKIILDNHEINFSGTLNGGNGFVSLTFSILEGINAVIEVDLLSKSYKVLISGELKTLMANSNSVQQKIDYIGLNSELQKNIPYSFMDDKGKENGFINCSTKEGLFVSELSDVVLISKVYMDNSKPLFGYCSNDLKDVKVITKDDVIILTGYYLKDDIKISLSFTIQDENTIKLNGVYLDENGVAEILKFMNKKGSTNTSDSLMSFLESMNIKSIFINSLQSNTKLILDTNFIISGTTSIGQFEFICDKDNNIQPYFIKFNTLETKYTLYVGNRQNMIVEPNYDLDDSGDISSTVINFSQKYLYGIDSCVNKVIISPNIYTDEINITPIYEANNTYPEVIVLDTNYISEKINININDLSIRYVWSNDGSDFILMSTDEENKVSQVKIRFTNVFKGNTISDKISFNFSDKQDVSINKVISTFTPSYYVEGLLNYDLGLISLYNEKFYINNFGMMVSGLVYINDSLYYFKPPIKNLITGFTTIGDDKYYFNPDNGGAASVGETIIDGKNYYFSQNGVLQTGVFSTEDGFKYFAPADTLDENLEGEAIDFTGKLTIDENVYYFGDNYRAAIEWQTLDDEVYYFSTDTGRAFKGLNQIGDDKFYFNSDGIMQKGFVNINDKTFYFDDSGVMKSGYTEIDGKYFYFAENGEMQIGVFNTADGFKYFAHHDEDLGNEEGEALSYSGILNFNNKIYYFDDSFTAVVGWKDLEDGSKYYFDEDTAEAYIGISIINDGKYYFNDSGIMQIGFVTINNEVFYFSDSGIVESGMQNIDDNYFYIDENGLVQIGVFDTSDGYKYFAPANTVNDNIYGQAVEYSGLVRVGEDVYYFGETYTIETGWIYDMENESDKYYFDPETKKAYKGINVIDDIKYYFDENGIMRTGLITFEDNHYYFNEDGIMQYGYLNIEDKTFYFSEDGIMQIGVFNTPDGFKYFAHQNTLDENFEGESINYTGWLDLDEKRYYFTDEYIAATGSVIIDGEEYYFDPDTAQLVISE

SEQ ID NO: 12 - protein, 2367 aa, Clostridioides difficile
  CHAIN 1..543: GTD domain
  Toxin B 1470 (clade 4)
  CHAIN 403..438: epitope
  CHAIN 544..767: CPD domain
  CHAIN 768..1852: TLD domain
  CHAIN 1853..2366: CROPs
  CHAIN 1875..2005: E1 domain
  CHAIN 2006..2137: E2 domain
  CHAIN 2138..2271: E3 domain
  CHAIN 2272..2366: E4 domain
MSLVNRKQLEKMANVRFRVQEDEYVAILDALEEYHNMSENTVVEKYLKLKDINSLTDTYIDTYKKSGRNKALKKFKEYLVIEILELKNSNLTPVEKNLHFIWIGGQINDTAINYINQWKDVNSDYNVNVFYDSNAFLINTLKKTIIESASNDTLESFRENLNDPEFNHTAFFRKRMQIIYDKQQNFINYYKAQKEENPDLIIDDIVKTYLSNEYSKDIDELNAYIEESLNKVTENSGNDVRNFEEFKTGEVFNLYEQESVERWNLAGASDILRVAILKNIGGVYLDVDMLPGIHPDLFKDINKPDSVKTAVDWEEMQLEAIMKHKEYIPEYTSKHFDTLDEEVQSSFESVLASKSDKSEIFLPLGDIEVSPLEVKIAFAKGSIINQALISAKDSYCSDLLIKQIQNRYKILNDTLGPIISQGNDFNTTMNNFGESLGAIANEENISFIAKIGSYLRVGFYPEANTTITLSGPTIYAGAYKDLLTFKEMSIDTSILSSELRNFEFPKVNISQATEQEKNSLWQFNEERAKIQFEEYKKNYFEGALGEDDNLDFSQNTVTDKEYLLEKISSSTKSSEGGYVHYIVQLQGDKISYEAACNLFAKNPYDSILFQRNIEDSEVAYYYNPTDSEIQEIDKYRIPDRISDRPKIKLTFIGHGKAEFNTDIFAGLDVDSLSSEIETAIGLAKEDISPKSIEINLLGCNMFSYSVNVEETYPGKLLLRVKDKVSELMPSMSQDSIIVSANQYEVRINSEGRRELLDHSGEWINKEESIIKDISSKEYISFNPKENKIIVKSKNLPELSTLLQEIRNNSNSSDIELEEKVMLAECEINVISNIETQVVEERIEEAKSLTSDSINYIKNEFKLIESISEALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKVMMQQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALPLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGSTNTSDSLMSFLESMNIKSIFVNFLQSNIKFILDANFIISGTTSIGQFEFICDENNNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFEDNYRGAVEWKELDGEMHYFSPETGKAFKGLNQIGDDKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFVPETKKACKGINLIDDIKYYFDEKGIMRTGLISFENNNYYFNENGEIQFGYINIEDKMFYFGEDGVMQIGVFNTPDGFKYFAHQNTLDENFEGESINYTGWLGLDEKRYYFTDEYIAATGSVIIDGEEYYFDPDTAQLVISE

SEQ ID NO: 13 - protein, 36 aa, Artificial Sequence
  Consensus Sequence
  MUTAGEN 3: X is E, Q, D, or N
  MUTAGEN 11: X is N, D, E, or Q
  MUTAGEN 12: X is S or T
  MUTAGEN 16: X is A, I, V, or L
  MUTAGEN 19: X is E, Q, D, or N
  MUTAGEN 20: X is D, G, A, V, I, or L
  MUTAGEN 27: X is T, M, or S
  MUTAGEN 29: X is T, A, N, S, T, L, V, I, or D
  MUTAGEN 31: X is I, G, L, V, or A
  MUTAGEN 32: X is D, E, N, or Q
  MUTAGEN 34: X is I, L, V, or A
  MUTAGEN 35: X is M, G, A, V, I, or L
QIXNRYKILNXXLNPXISXXNDFNTTXNXFXXSXXA